(19) United States
(12) Patent Application Publication
(10) Pub. No.: US 2002/0077557 A1
(43) Pub. Date: Jun. 20, 2002

(54) Method of periodically or constantly watching a person's blood pressure and system thereof

(75) Inventor: Hui-Wen Cheng, Taipei (TW)

Correspondence Address: BACON & THOMAS, PLLC, 625 Slaters Lane - 4th Floor, Alexandria, VA, 22314, US

(73) Assignee: TrustMed. Com Corp., Taipei (TW)

(21) Appl. No.: 09/781,970

(22) Filed: Feb. 14, 2001

(30) Foreign Application Priority Data
Oct. 31, 2000 (TW) 89122928

(51) Int. Cl. A61B 5/00

(52) U.S. Cl. 600/490; 600/300; 705/3

(57) ABSTRACT

A system of watching a person's blood pressure includes a database server; a network server; and a communication devices; wherein the database server is built-in with a database of blood pressure and with a software, so that a) the database server determines time for a person to measure his/her blood pressure according to medical criteria on his/her blood pressure history recorded in the database; b) the network server and the communication device notify the person to measure his/her blood pressure in advance the determined time and report his/her measured blood pressure to the network server according to the determined time; and c) the network server and the communication device remind the person of the blood pressure measurement if the network server should fail to receive his/her measured blood pressure after the determined time.

### FIELD OF THE INVENTION

The present invention relates to a method and a system of periodically or constantly watching a person's blood pressure.

### BACKGROUND OF THE INVENTION

After receiving a treatment from a doctor, a hypertensive patient should continue his/her treatment according to the appointment with the doctor. However, due to lack of an adequate reminding system, the hospital is unable to remind a patient of the appointment in advance or after the patient misses the appointment. Therefore, a hypertensive patient suffers a higher risk of seizure or death from cardiovascular disease.

### SUMMARY OF THE INVENTION

The present invention provides a method and a system of periodically or constantly recording and watching a patient's blood pressure to solve the above-mentioned problems long existed in the medical field.

In one aspect of the present invention, a method of periodically or constantly recording and watching a patient's blood pressure according to the present invention is carried out via a network.

In another aspect of the present invention, a system of periodically or constantly recording and watching a patient's blood pressure according to the present invention works cooperatively with a network.

### BRIEF DESCRIPTION OF THE DRAWINGS

FIG. 1 is a flowchart showing a method of periodically or constantly recording and watching a patient's blood pressure according to a first embodiment of the present invention.

FIG. 2 is a schematic block diagram showing a system of periodically or constantly recording and watching a patient's blood pressure according to the present invention.

FIG. 3 is a flowchart showing a method of periodically or constantly recording and watching a patient's blood pressure according to a second embodiment of the present invention.

FIG. 4 is a flowchart comprising steps for deciding whether a client is a new hypertensive patient or not based on the blood pressure data.

### DETAILED DESCRIPTION OF THE INVENTION

The present invention discloses a method of periodically or constantly watching a person's blood pressure comprising the following steps carried out in a computer system:

a) determining time for a person to measure his/her blood pressure according to medical criteria on his/her blood pressure history recorded in a database of blood pressure of persons;

b) notifying said person to measure his/her blood pressure and report his/her measured blood pressure to said computer system in advance the determined time;

c) reminding said person of the blood pressure measurement if said computer system should fail to receive his/her measured blood pressure after the determined time; and

d) entering the measured data of blood pressure of said person into the database of blood pressure of persons, if said computer system receives his/her measured blood pressure.

Preferably, said medical criteria are:

once per two years if the blood pressure<130/85;

once per year if 130/85≦the blood pressure<140/90;

once per two months if 140/90≦the blood pressure<160/100;

once per month if 160/100≦the blood pressure<180/110; and

once per two weeks if the blood pressure≧180/110.

Preferably, said reminding is once per day.

Preferably, said notifying or said reminding step adopt a WAP system.

The database of blood pressure is preferably set up before the method of the present invention is implemented; however, it can also be set up when the method of the present invention is initiated.

The database of blood pressure contains data of a hypertensive or a potential hypertensive patient that should be recorded by a medical practitioner, e.g. name (or ID number, such as citizen's ID number, number of registration card of a hospital), data of blood pressure, etc., preferably may include further detailed information of the patient, e.g. habits of living, and risk factors that might cause hypertension, e.g. smoking, high blood-lipid, diabetes, family history of high blood pressure, personal history of high blood pressure, personal history of heart disease, history of kidney disease, etc.

The medical criteria for determining the time for a patient to measure his/her blood pressure can be a doctor's subjective judgement or according to the general regulation of once per month for a hypertensive patient.

The diagnostic of blood pressure can be carried out according to the subjective judgement by a doctor, from a specific checklist or medical criteria (as shown in Table 1).

TABLE I

| | | |
|---|---|---|
| BP < 130/85 | once per two years | Blood pressure is |
| | | normal |
| 130/85 ≦ BP < 140/90* | once per year | Blood pressure is |
| | | normal but slightly |
| | | higher |
| 140/90 ≦ BP < 160/100 | once per two months | Slightly hypertension |
| 160/100 ≦ BP < 180/110 | once per month | Moderate hypertension |
| BP ≦ 180/110 | once per two weeks | Severe hypertension |

*BP = blood pressure, and 130/85 ≦ BP < 140/90 means that the systolic pressure is not less than 130 and the diastolic threshold is not less than 85; the systolic pressure is smaller than 140 and the diastolic threshold is smaller than 90.

The above-mentioned notification or reminder can be carried out by using any known methods of notification or reminder, such as notification/reminder by a conventional telephone, notification/reminder through a network (e.g. e-mail, mobile phone, cable telephone, etc.) Preferably, the notification/reminder is carried out through a network. More preferably, the notification/reminder is carried out by using a mobile phone through a network. Most preferably, the notification/reminder is carried out by a mobile phone system using WAP (Wireless Application Protocol).

The abovementioned reminder is based on whether a hypertensive patient has reported the measured blood pressure within a certain period of time after receiving a notification (or a previous reminder). Of course, the number of times of reminding can also be set to a default value, e.g. stop reminding, when no response from the patient is received after a specified number of reminders.

Other data related to blood pressure, e.g. change of lifestyle of a hypertensive patient and other history of related diseases, can also be simultaneously recorded in the database.

The term “in advance” varies according to the need. For example, a patient reserving a clinical visit requires only slightly earlier (a few days or even earlier that day); a patient without a reservation requires a much earlier notification for the patient to register or make a reservation.

The present invention also discloses a system of watching a person's blood pressure comprising:

a database server;

a network server; and

communication means;

wherein said database server is built-in with a database of blood pressure and with a software, so that

A) said database server determines time for a person to measure his/her blood pressure according to medical criteria on his/her blood pressure history recorded in said database;

B) said network server and said communication means notify said person to measure his/her blood pressure in advance the determined time and report his/her measured blood pressure to said network server according to the determined time; and

C) said network server and said communication means remind said person of the blood pressure measurement if said network server should fail to receive his/her measured blood pressure after the determined time.

The database server used in the system of the present invention can be any known database servers.

The network server can be any known network servers, and said communication means can adopt any known communication means, preferably wireless communication means, more preferably a WAP system.

Said determination can be a regular determination or a random determination. Preferably, the determination is carried out after each recordal of new measurement of blood pressure in order to save the time spent on the determination.

The content of notification can be a simple date of measurement of blood pressure, or can be added with other contents, such as common sense/knowledge/information related to blood pressure measurement or control of hypertension, etc.

The method and the system of periodically or constantly recording and watching blood pressure according to the present invention is not limited to be used by a hospital, and can be used by a medical research organization or an organization in the medical profession having a power of public reputation, and/or a subsidiary thereof.

In order to further elaborate the present invention, preferred embodiments are illustrated together with figures. However, the scope of the present invention is not limited by the preferred embodiments or figures.

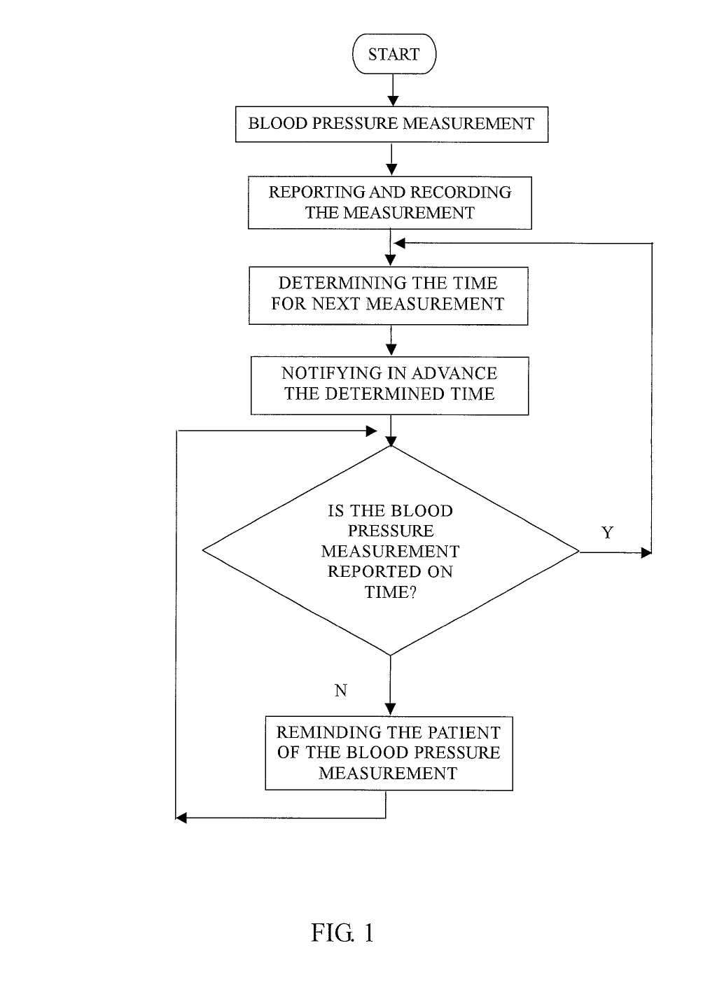

FIG. 1 is a schematic flowchart of a method of periodically recording and watching blood pressure implemented in a hospital. The method comprises measuring a patient's the blood pressure during a clinical visit; recording and reporting the measured blood pressure; determining the time for next measurement; notifying the patient in advance the determined time; checking whether the patient reports his/her blood pressure according to the determined time; reminding the patient of the blood pressure measurement, if the answer is “No”, and returning to the step of determining the time for next measurement, if the answer is “Yes”.

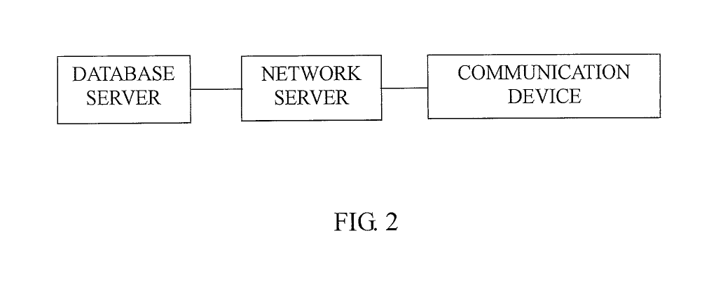

FIG. 2 is a block diagram of a system of periodically recording and watching blood pressure for use in a hospital, in which the database server uses a computer and a UNIX system, the network server uses a computer and a UNIX system, and the communication device uses a WAP system. The database server and the communication device are connected to the network server.

A new patient only needs to enter the required data to the database of blood pressure before, after or at the time of measuring the blood pressure.

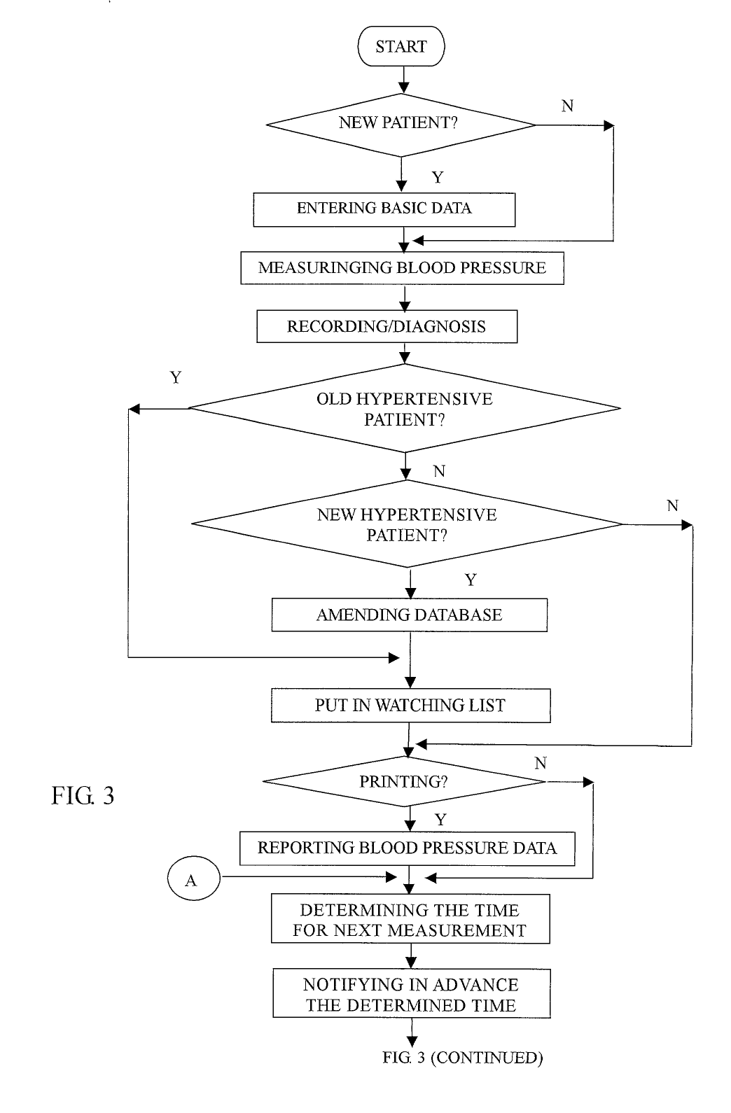

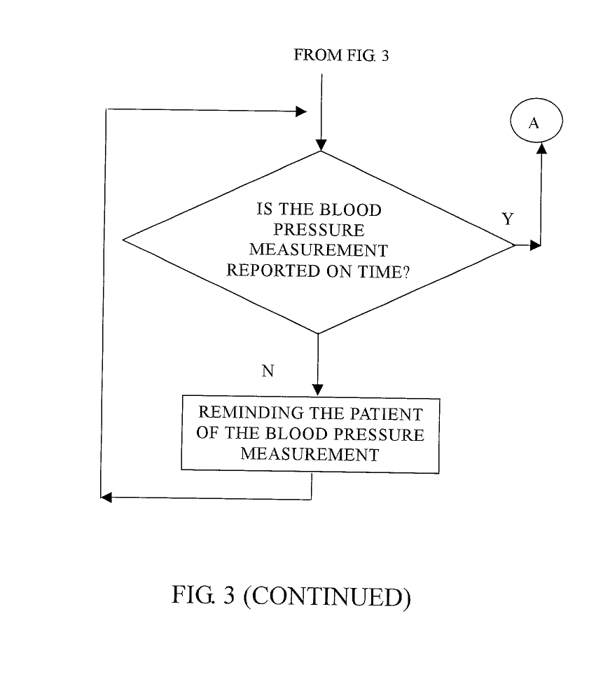

In another embodiment, the method of the present invention includes setting up the database for new patients. As shown in FIG. 3, the method of this embodiment is similar to that shown in FIG. 1, with the addition of three determination formulas: “whether this is a new client (patient)?”, “whether this is an old hypertensive patient?”, and “whether this is a new hypertensive patient?”. The method according to this embodiment first decides whether a person taking a blood pressure measurement is a new client (patient) or not. A new patient is required to enter the basic data before the measurement. The blood pressure measurement is then recorded and is used for diagnosis. The status of the client will be checked to decide whether the client is an old hypertensive patient or not. If the client is not an old hypertensive patient, he/she will be decided as a new hypertensive patient or not based on the blood pressure data. If the answer is “Yes”, the status of the client will be changed to a hypertensive patient and put into the watching list. If the answer is “No”, the client will not be put into the watching list. If the client is an old hypertensive patient, he/she will be put in the watching list directly. The method will then decide whether the blood pressure data of the client is to be printed, so that the client can receive the printing report which may include the historical data of blood pressure, the trend diagram of blood pressure, etc. The steps thereafter are same as those shown in FIG. 1.

The printing report may further include the data listed in Table 1 and the suggestions for controlling the hypertension listed in Table 2.

TABLE 2

| Blood pressure | Group A** | Group B** | Group C** |
|---|---|---|---|
| 130/85 ≦ BP | Adjusting life | Adjusting life | Should receive |
| < 140/90* | style | style | medical |
| | | | treatment |
| 140/90 ≦ BP | Adjusting life | Adjusting life | Should receive |
| < 160/100 | style for a year | style for six | medical |
| | | months | treatment |
| BP ≦ 160/100 | Should receive | Should receive | Should receive |
| | medical | medical | medical |
| | treatment | treatment | treatment |

*Same as Table 1.
**Group A: no major risk factors, no damaged target organs or clinical cardiovascular diseases; Group B: at least one major risk factor, no diabetes, no damaged target organs or clinical cardiovascular diseases; and Group C: has damaged target organ(s) or clinical cardiovascular disease(s) or diabetes, major risk factors are optional.

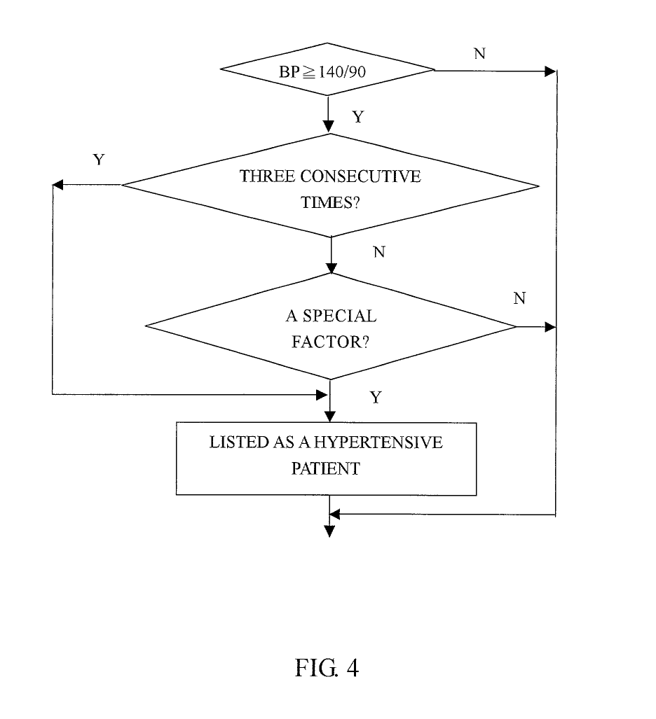

In FIG. 3, the step of deciding whether the client is a new hypertensive patient or not based on the blood pressure data may comprise the sub-steps shown in FIG. 4: checking whether the blood pressure of the client is equal to or greater than 140/90; further checking whether the blood pressure of the client is equal to or greater than 140/90 for three consecutive measurements when the answer is “Yes”; and checking whether there is a special factor to be considered, e.g. cardiovascular disease, if only one or two measurements of blood pressure are higher than 140/90. The client will be listed as a hypertensive patient if three consecutive measurements of blood pressure are higher than 140/90, or if one to two measurements of blood pressure are higher than 140/90 with the special factor.

What is claimed is:

1. A method of periodically or constantly watching a person's blood pressure comprising the following steps carried out in a computer system:
a) determining time for a person to measure his/her blood pressure according to medical criteria on his/her blood pressure history recorded in a database of blood pressure of persons;
b) notifying said person to measure his/her blood pressure and report his/her measured blood pressure to said computer system in advance the determined time;
c) reminding said person of the blood pressure measurement if said computer system should fail to receive his/her measured blood pressure after the determined time; and
d) entering the measured data of blood pressure of said person into the database of blood pressure of persons, if said computer system receives his/her measured blood pressure.

2. The method as claimed in claim 1, in which said medical criteria in step a) are:
once per two years if the blood pressure<130/85;
once per year if 130/85≦the blood pressure<140/90;
once per two months if 140/90≦the blood pressure<160/100;
once per month if 160/100≦the blood pressure<180/110; and
once per two weeks if the blood pressure≧180/110.

3. The method as claimed in claim 1, in which said reminding is once per day.

4. The method as claimed in claim 17 in which said notifying or said reminding step adopt a WAP system.

5. A system of watching a person's blood pressure comprising:
a database server;
a network server; and
communication means;
wherein said database server is built-in with a database of blood pressure and with a software, so that
a) said database server determines time for a person to measure his/her blood pressure according to medical criteria on his/her blood pressure history recorded in said database;
b) said network server and said communication means notify said person to measure his/her blood pressure in advance the determined time and report his/her measured blood pressure to said network server according to the determined time; and
c) said network server and said communication means remind said person of the blood pressure measurement if said network server should fail to receive his/her measured blood pressure after the determined time.

6. The system as claimed in claim 5, in which said medical criteria are:
once per two years if the blood pressure<130/85;
once per year if 130/85≦the blood pressure<140/90;
once per two months if 140/90≦the blood pressure<160/100;
once per month if 160/100≦the blood pressure<180/110; and
once per two weeks if the blood pressure≧180/110.

7. The system as claimed in claim 5, in which said communication means comprises a WAP system.
